(19) United States
(12) Patent Application Publication
(10) Pub. No.: US 2006/0126018 A1
(43) Pub. Date: Jun. 15, 2006

(54) Methods and apparatus for wavefront sensing of human eyes

(76) Inventor: Junzhong Liang, Fremont, CA (US)

Correspondence Address: XIN WEN, 3449 RAMBOW DRIVE, PALO ALTO, CA, 94306, US

(21) Appl. No.: 11/293,611

(22) Filed: Dec. 2, 2005

Related U.S. Application Data
(60) Provisional application No. 60/635,248, filed on Dec. 10, 2004.

(51) Int. Cl. A61B 3/10

(52) U.S. Cl. 351/211; 351/205; 351/221

(57) ABSTRACT

A wavefront sensing system for determining the wave aberration of an eye comprises a fixation target configured to keep the eye focus at its far accommodation point by illuminating the fixation target with a light source at a location optically conjugate to the cornea of the eye, an illumination light source configured to produce a compact light source at the retina of the eye, and a wavefront sensor configured to measure the outgoing wavefront originated from the compact light source at the retina. The compact light source at the retina of the eye in the wavefront sensing system is obtained by illuminating the cornea of the eye with a fixed divergent beam that is optimized for a normal population without the need of a refractive correction for the focus error and astigmatism. The outgoing wavefront originated from the compact light source at the retina is refracted by a cylindrical lens before being measured if the wavefront sensor is a Hartmann-Shack sensor. The wavefront sensing system can include a non-contact opto-sensor configured to detect the left and the right eye automatically during a wavefront measurement.

### CROSS-REFERENCES TO RELATED INVENTIONS

The present invention claims priority to the provisional U.S. patent application 60/635,248, titled “Methods and apparatus for wavefront refraction system” filed on Dec. 10, 2004 by Liang. The present invention is related to commonly assigned and concurrently filed U.S. patent application “Improved methods and systems for wavefront analysis” filed by Liang et al. The disclosures of these related applications are incorporated herein by reference.

### TECHNICAL FIELD

This application relates to systems and methods for measuring human vision, in particular, the wavefront sensing of human eyes.

### BACKGROUND

Wavefront-guided vision correction is becoming a new frontier for vision and ophthalmology. It offers supernormal vision beyond conventional sphero-cylindrical correction, allowing the imaging of living photoreceptors and the perfection of laser vision correction. Wavefront technology will reshape the eye care industry by enabling customized design of laser vision correction, contact lenses, intro-ocular lenses, and even spectacles. The first precise method for the detection of wave aberrations was disclosed in “Objective measurement of wave aberration of the human eye with the use of a Hartmann-Shack wave-front sensor” J. Opt. Soc. Am. A, vol. 11, no. 7, p. 1949, by Liang et al., in July, 1994. A typical wavefront sensing system for the eye consists of a fixation target, a probing light illumination, and a wavefront sensor such as a Hartmann-Shack sensor. Wave aberration represents all aberrations including nearsightedness (farsightedness), astigmatism, coma, spherical aberrations and a host of other irregular aberrations.

The fixation target in a wavefront refractor provides visual stimuli to the tested eye. The fixation target ensures the tested eye to focus at its far accommodation point. Conventional fixation designs use a large uncontrolled pupil and have several disadvantages. First, moving optical components is often required for measuring eyes with different refractive corrections. A moving fixation system requires expensive components and prolongs measurement time. Keeping the eye wide open for a long period during the measurement can be rather uncomfortable for the patient. Second, conventional fixation targets without a dynamic focus correction are hardly visible when the tested eye has high refractive correction beyond a few Dioptors. A need clearly exists in the art for an improved fixation target that is low cost and can comfort to the patient.

Focus error or the spherical correction (myopia or hyperopia) is the largest refractive error in the eye. For the vast majority of the population that needs a vision correction, the spherical correction is in the range between −12 D (myopia) and +6 D (hyperopia). If uncorrected, the focus error in the eye can cause the formation of a severely blurred light spot at the retina which makes it not suitable for wavefront sensing. Conventional wavefront refractors use an optical system to dynamically correct eye's focus error to produce a compact light source on the retina. U.S. Pat. No. 6,736,509 by Martino describes an illumination approach that eliminates the dynamic focus correction between the light source and the patient cornea. Martino illuminates a collimated light beam at the cornea to produce a diffraction-limited light spot at the retina. Martino's solution is however not optimized. First, a collimated beam illuminating at the cornea is off balance for the vision-correction population that is biased towards myopia. Second, wavefront sensors only require a compact light source at the retina rather than a diffracted-limited retinal image. A need clearly exists in the art to further optimize the illumination for the wavefront sensor without the need of correcting eye's sphero-cylindrical corrections.

A Hartmann-Shack sensor contains a lenslet array and an image sensor. The lenslet array divides the measured wavefront into a number of subapertures and produces an array of focus spot at the focal plane of the lenslets. The image sensor is often placed at the focal plane of the lenslet array and converts the light signal to an digital image. Using commercial video image sensors is prefered for low-cost wavefront systems but limited because the test opatical zone has a circur shap whereas the video image sensor has a rectangular photosensitge area. A need exists in the art to develop an effective mean for the best use of a small rectangular video sensor for wavefront sensing of a nearly circular area.

Another need for the wavefront sensing for the eye is to identify the left eye (OS) and the right eye (OD) automatically in the wavefront measurement. A mismatch of wavefront measurement data for the left and right eye of a patient can cause incorrect treatments, which must be prevented.

### SUMMARY

The present invention is directed to a wavefront sensing system for determining the wave aberration of an eye, comprising:

a fixation target that is illuminated by a fixation light source at a location optically conjugate to the cornea of the eye, wherein the fixation target is partially visible by the eye without the need of a refractive correction and configured to keep the eye focus at its far accommodation point;

an illumination light source configured to produce a compact light source at the retina of the eye; and

a wavefront sensor configured to measure the outgoing wavefront originated from the compact light source at the retina of the eye to determine the wave aberration of the eye.

In another aspect, the present invention includes an optimized illumination light source for the design of a wavefront sensor without an refractive correction for myopia, hyperopia or astigmatism, comprising an fixed divergent light beam through the pupil of the tested eye and the illumination beam is configured to produce a compact light source at the retina according to a criterion of one-half wavelength.

In still another aspect, the present invention includes an improved design of a Hartmann-Shack sensor comprising a lenslet array, a cost-effective rectangular image sensor, and a cylindrical lens to refract the tested wavefront before being measured.

In yet another aspect, the present invention includes an illumination light configured to illuminate a plurality of locations at the pupil of the eye to produce a compact light source at the retina of the eye sequentially.

In another aspect, the present invention includes a non-contact sensor for the detection of left and right eye in a wavefront sensing system.

Embodiments may include one or more of the following advantages. The invention system provides improved and cost-effecitve measurements of eye's wave aberration using wavefront sesnsing techniques.

The invention system provides a cost effective fixation target in wavefront sensor devices. The fixation target ensures the tested eye to accommodate its viewing at its far point during measurement without a moving part. The measurment time is significantly reduced for the comfort of patients.

Another advantage of the present invention is that it optimizes the illumination beam in a wavefront sensing system to remove the need of a dynamic correction of the sphero-cylindrical corrections of the tested eye for the majority of the vision-correction population.

Another advantage of the invention system is that it provides a relaxed creterion for the retinal illumination for wavefront sensing devices that takes into account of eye's refractive errors. A more tolerant criterion allows eyes with significantly larger focus error to be measured in comparison to use the conventional diffraction-limited criterion.

Still another advantage of the invention system is that it provides an inexpensive design for the image sensor in a wavefront sensing system for humane eyes.

Yet another advantage of the invention system is that it provides a cost-effective, non-contact, and automatic detection of the left and right eye in the wavefront measurements, which eliminates the chance of mismatching the wavefront measurement result for a patient's left and right eyes.

The details of one or more embodiments are set forth in the accompanying drawings and in the description below. Other features, objects, and advantages of the invention will become apparent from the description and drawings, and from the claims.

### DRAWING DESCRIPTIONS

FIG. 1 shows a schematic diagram of a wavefront sensing system in accordance with the present invention.

FIG. 2a is a schematic diagram for a conventional fixation system for a wavefront refractor.

FIG. 2b illustrates an improved fixation system compatible with the wavefront sensing system in FIG. 1 in accordance with the present invention.

FIG. 3a illustrates the distribution of retinal point spread function with a circular pupil.

FIG. 3b illustrates the one dimensional profiles of point-spread function with focus error measured in term of peak-to-valley wavefront error.

FIG. 4a shows optimization of the probing light in a wavefront sensor of an eye using a divergent beam.

FIG. 4b illustrates the retinal point-spread distributions for an eye with focus error of +6 D, 0 D, −6 D and −12 D using a narrow and (−3 D) divergent beam at the cornea.

FIG. 5a shows a conventional wavefront sensor with a square lenslet array and a rectangular image sensor.

FIG. 5b shows a wavefront sensor image having focus spots distributed outside the rectangular image sensor of FIG. 5a.

FIG. 5c illustrates a wavefront sensor having a cylindrical lens in front of the lenslet array and a rectangular video image sensor.

FIG. 5d illustrates the reduction of wavefront image along the short axis by the cylindrical lens.

FIG. 6a show a non-contact optical sensor for automatic detection of left and right eye.

FIG. 6b shows the voltage output of the optical sensor of FIG. 6a to specify the right eye and the left eye.

FIG. 7 illustrates the configurations of moving the probing light beam for wavefront sensing measurement.

FIG. 8a shows an arrangement for scanning a small illustration beam across the pupil of a tested eye in one direction.

FIG. 8b shows an arrangement for rotating a small illustration beam across the pupil of a tested eye.

### DETAILED DESCRIPTION

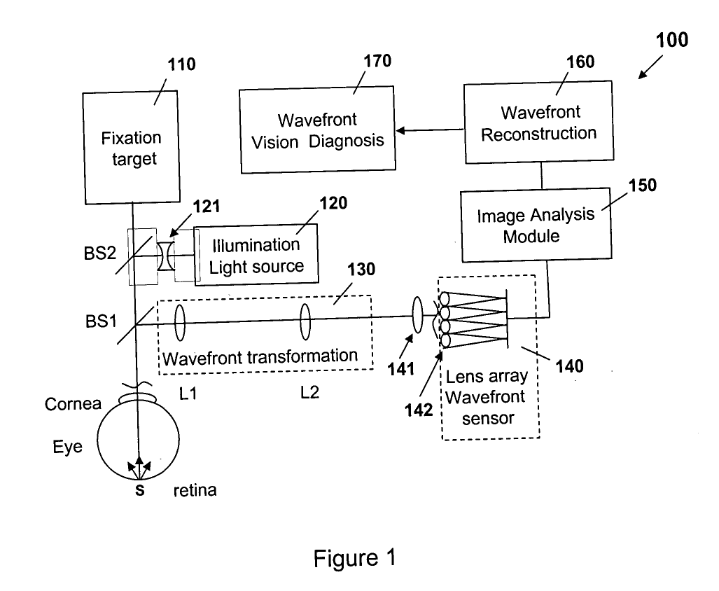

FIG. 1 illustrates a schematic diagram of a wavefront-sensing system 100 in accordance with the present invention. A fixation system 110 is provided to stabilize the tested eye for accommodation control (i.e. the control of the eye's focus position). A collimated light source 120 is converted to a small divergent beam of light by a negative spherical lens 121. The divergent beam is reflected off a beam splitter (BS2) and generates a compact light source (S) at the retina of the eye. The compact light beam illuminates the eye's retina and is diffusely reflected by the retina. The illumination light beam for the compact light source at the retina is referred as the probing light beam or the illumination light beam. The reflected light propagates to the eye's cornea and forms a distorted wavefront at the cornea plane. The distorted wavefront is reflected off a beam splitter BS1 and then relayed by an optical relay system 130 to a Hartmann-Shack wavefront sensor 140. The optical relay system 130 consists of lenses (L1) and (L2). A cylindrical lens 141 introduces a fixed cylindrical wave to the wavefront from the eye before it enters Hartmann-Shack wavefront sensor 140. The Hartmann-Shack wavefront sensor 140 includes a lenslet array 142 and an image sensor. The lenslet array 142 converts the distorted wavefront to an array of focus spots on the image sensor. An image analysis module 150 detects the focus spots and calculates the slopes of the wavefront. A wavefront estimator 160 reconstructs the wavefront using the slopes of the wavefront. A vision diagnosis module 170 determines the eye's optical quality and optical defects, which can provide the basis for a vision correction diagnosis.

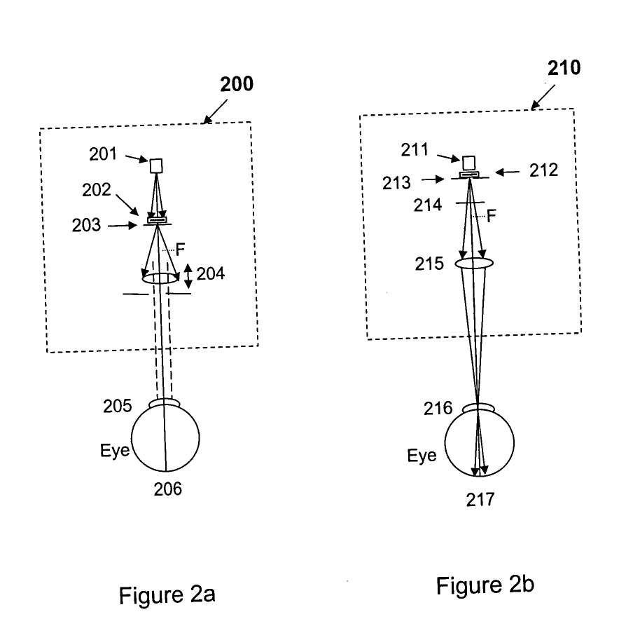

FIG. 2a shows a schematic diagram of a conventional fixation system 200 using incoherent light and an uncontrolled pupil size. A target object 203 is illuminated by an incoherent light source 201 through a diffuser 202. A lens 204 is placed next to the target object 203. The distance between the target object 203 and the lens 204 is adjustable. At the beginning of a measurement, the lens 204 is located at a distance relative to the target object 203 such that the target object 203 is out of focus to the eye. The lens 204 is then moved toward the object to bring the target object 203 in focus to the retina of the eye (206) through the optics of the eye (205). In order to make the tested eye accommodate at its far point, the lens can be moved away from the target object 203 at the final stage of the measurement to bring the target object 203 out of focus again to the eye. The last defocusing movement is often referred to as fogging.

FIG. 2b shows an improved fixation system 210 compatible with a wavefront-sensing system 100 in accordance with the present invention. A small pinhole aperture 213 is set optically conjugate to the cornea plane 216. The pinhole aperture 213 is illuminated by a light emitting diode (LED) 211 through a diffuser 212 providing a spatially coherent light source. The target object 214 is illuminated by the spatially coherent light from the pinhole aperture 213. The target object 214 is then imaged on the retina 217 of the eye through lens 215 and the lens optics 216 of the eye. The target object 214 is set at a fixed position throughout the measurement. There is no moving part in the wavefront measurement. For the normal population, the target object 214 is set at +6 D (Diopters) for the emmotropic eyes. The target object 214 contains broad spatial frequencies up to 60 cycles/deg like a Siemens Section Star.

The conventional fixation system 200 uses an incoherent light source and the entire pupil of the eye for all spatial frequency. In contrast, the improved fixation system 210 uses a coherent light source and coherent imaging system, in which different effective pupil sizes are used for different spatial frequencies. The distribution of the illumination light near the eye's cornea is the Fourier spectra of the fixation target. The low frequency components of the fixation target are distributed at the center of the pupil whereas the high frequency components are away from the pupil center. Therefore, the high spatial frequencies use a large effective pupil size and are more sensitive to focus error. Low spatial frequencies use a smaller effective pupil size. The use of a smaller effective pupil size yields also a large depth of focus.

The improved fixation target system 210 includes the following advantageous features. First, the fixation target is out of focus for the tested eyes from the near point to the far point because the fixation target is in focus only for hyperopic eyes at +6 D. This is important for the tested eye to try to accommodate at its far point for the best image quality available. Second, the wave aberration of the eye is measured at its far focus point because the tested eye has the best image quality when the eye accommodated at its far point. Third, a significant portion of the fixation target is always visible because of small effective pupil for low spatial frequencies and long depth of focus. Visible fixation target prevents measuring eye's wave aberration at a random different focus state. Finally, the improved fixation target system 210 contains no moving part, which allows for instant wavefront measurement and leads to a low cost system.

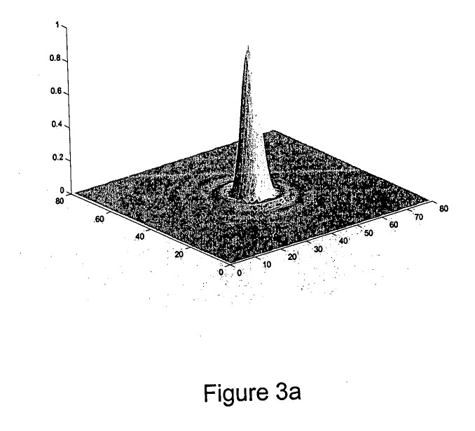

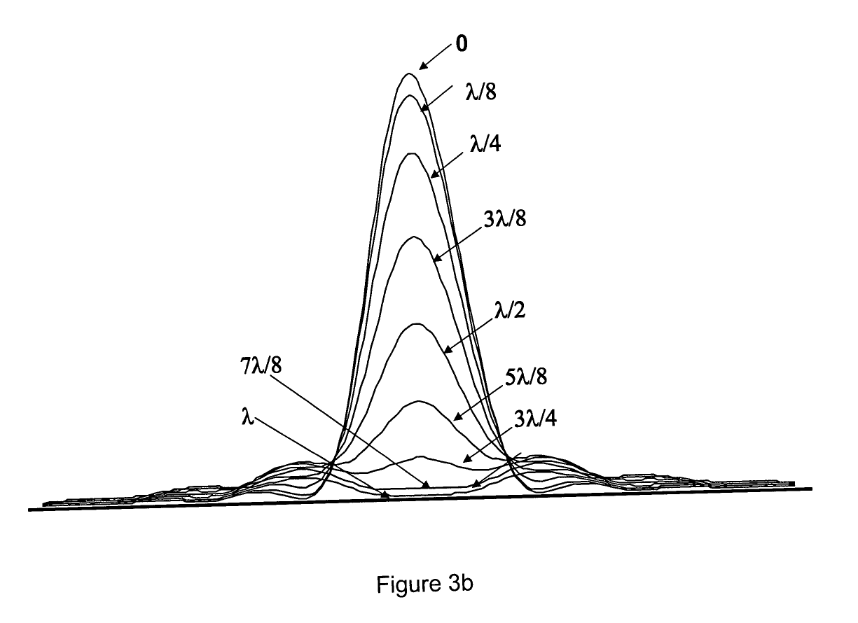

Wavefront sensor for the eye requires a compact light source at the retina. FIG. 3a shows a retinal point-spread function with a small amount of focus error. The distribution of the point image is center symmetric. FIG. 3b shows the normalized profiles of the point-spread functions for different amounts of focus errors. When the focus error is within a ¼ wave, the point spread function appears to be diffraction-limited containing a strong central peak and much weaker side-peaks. As wavefront error is increased, the central peak decreases whereas the side peaks increase. The central peak becomes lower than the side peak when the focus error is greater than a ¾ wavelength. The retinal point-spread function is compact with a strong central peak and much weaker side peaks within ½ wave focus error as seen in FIG. 3b. Therefore, the threshold for the acceptable wavefront errors is chosen to be less than ½ wave. By relaxing the threshold for the wavefront error from ¼ wavelength for diffraction-limited imaging to ½ wavelength for a compact distribution, a given probing light beam can be used to measure eyes having twice the focus range for a fixed aperture.

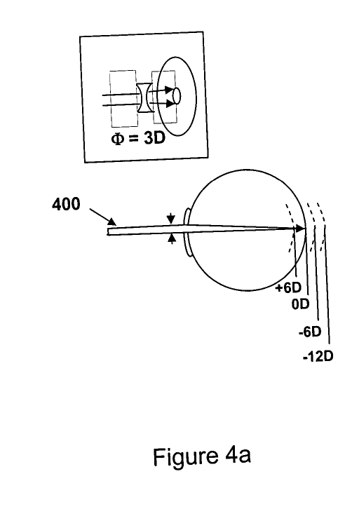

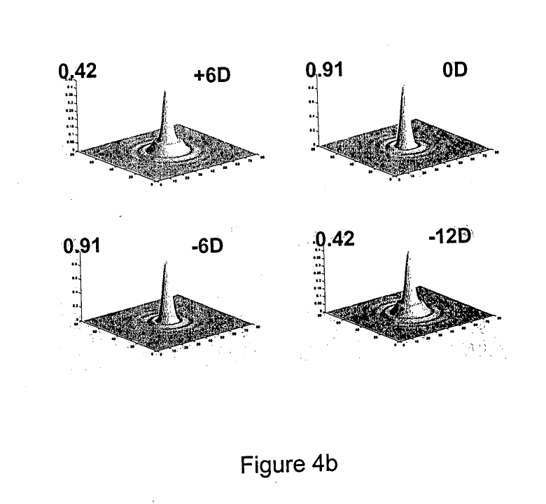

FIG. 4a shows the characteristics of a probing light beam on the retina in accordance with the present invention. A divergent beam 400 of −3 D rather than a collimated beam is used to illuminate the corneal plane. The divergent beam 400 is kept fixed during the measurement. The beam is uniform and approximately 0.6 mm in diameter at the corneal plane. The probing light beam is designed to be located at the center of the normal population with naked-eye refractive error ranging from −12 D to +6 D. FIG. 4b shows the simulated point spread functions of the probing beam 400 illustrated in FIG. 4a. The wavefront error within the illumination beam is one half wavelength for an eye with a spherical correction of +6 D and −12 D, and one quarter wavelength for an eye with a spherical correction of 0 D and −6 D. Compact light sources can be formed at the retina of the eye having spherical refractive errors of +6 D, 0 D, −6 D and −12 D.

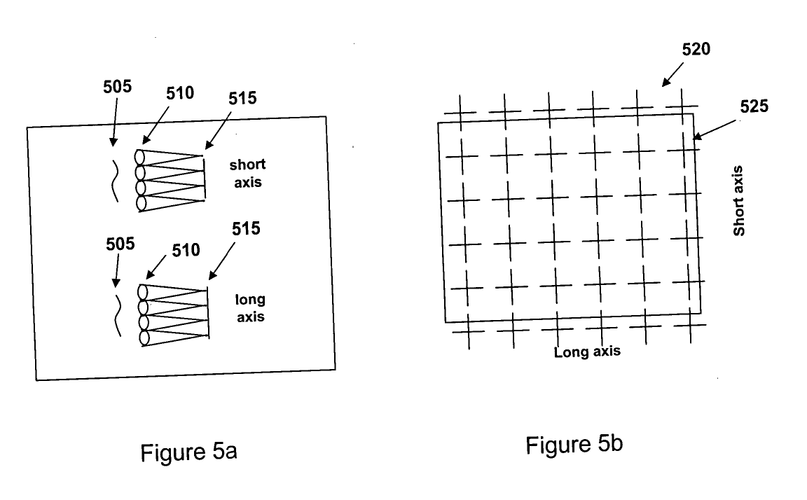

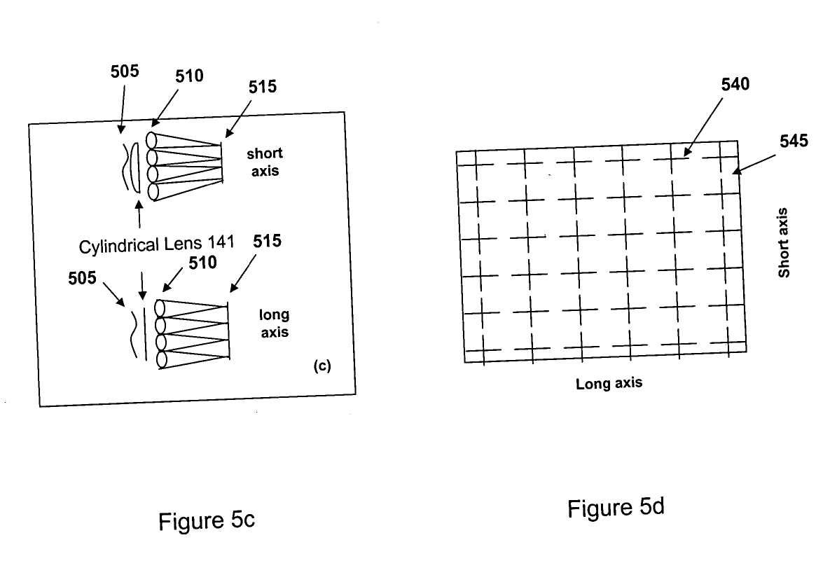

The wavefront sensor for the eye measures aberrations of the eye by sensing the outgoing wavefront originated from a compact light source at the retina. FIG. 5a shows wavefront measurements with a lenslet array 510 in a conventional wavefront sensing system. The distorted wavefront 505 is focused by the lenslet array 510 on the image plane 515. The focus spots 520 are formed by the lenslet array 510, which are distributed in an approximate square pattern as the lenslet array. An image sensor 525 is positioned at the image plane to capture the image of the focus spots. Since low-cost image sensors 525 are typically of a rectangular shape with an aspect ratio of 3 to 4, the focus spots 520 may be distributed outside of the image sensor 525 along the short axis of the image sensor 525, as shown in FIG. 5b.

As shown in FIG. 1 and FIG. 5c, a positive cylindrical lens 141 is placed in front of the wavefront sensor in accordance with the present invention. The cylinder lens 141 reduces the length of the image along the short axis of the image sensor 545, but does not change the spot pattern in the long axis. The cylindrical lens 141 can be placed at an optically conjugate position relative to the lenslet array 510. The cylindrical lens 141 thus produces a rectangular distribution of focus spots 540 to better match a rectangular image sensor 545, as shown in FIG. 5d. The use of the rectangular-shaped video image sensor 545 can significantly reduce the cost for wavefront sensing of eye.

Proper selection of the cylindrical lens is important in using a rectangular image sensor. First, the power of the cylinder lens should be properly chosen so that the astigmatism induced by the cylindrical lens within each lenslet is less than ¼ to ⅛ wavelength. The astigmatism in each lenslet is given by
W=Φₐd²/8
where Φₐ is the cylindrical power of the cylindrical lens in Diopters and d is aperture size of each lenslet. Second, the reduction of wavefront sensor image along the short axis is represented by
D=2*x*f*/fₐ
where x is radius of eye's pupil, f the focal length of the lenslet, and fₐ is the focal length of the cylindrical lens. If x=3.5 mm, f=40 mm, fₐ=165 mm, the reduction along the short axis is 1.5 mm. For a ⅔ inch camera, the long axis and short axis are 6.6 mm by 8.8 mm, respectively. A reduction of 1.5 mm in the short axis increase the chip size effectively to 8.1 mm from 8.8 mm, which is significant for using inexpensive CCD chips.

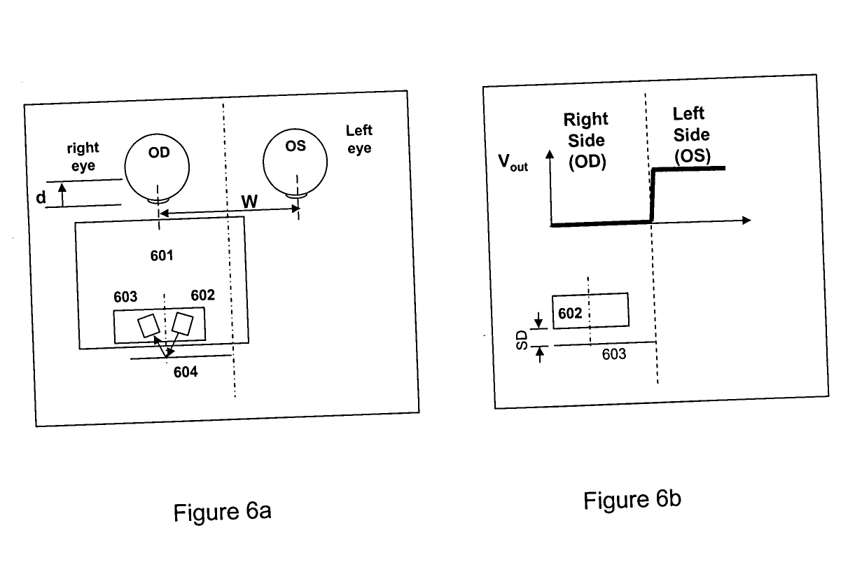

FIG. 6a illustrates a schematic diagram of a non-contact OD/OS sensor for left and right eye of a patient. An optical sensor 601 consists of a LED light source 602 and a photo-detector 603. The optical sensor 601 is mounted together with the movable wave sensing system, which can be moved between a left-eye measurement position and a right-eye measurement position. A reflective object 604 is mounted on a stationary base on the right eye side. The optical sensor 601 needs to have proper range of sensing distance (SD) to handle the variation in the vertical distance between the eyes and the chin. During a wavefront measurement, a patient's head is usually supported by a chin-rest and head-rest so that the tested eye is fixed in the air. The exact corneal location of the tested eye can be different from eye to eye. In order to measure the wavefront at the corneal plane, the wavefront sensor system together with the optical sensor 601 must be moved towards or away from the tested eye. Since the reflective object is fixed on the stationary base, the optical sensor 601 is required to function in a sensing distance (SD) between 5 mm to 30 mm.

When the optical sensor 601 is at the right-eye measurement position, the light from the LED light source 602 is reflected off the reflective target 604 and sensed by the photo detector 603. The optical sensor 601 outputs a logic “0” as shown in FIG. 6b. When the optical sensor 601 is moved to the left-eye measurement position, no light signal is detected by the photo detector 603. The photo sensor outputs a logic “1”. The eye position information is recorded to specify the wavefront sensing data collected from each eye to prevent mismatch of wavefront sensing data from the two eyes.

In another embodiment, the probing light beam is designed to be moved within the eye's pupil to avoid potential anomalous locations in the optics of the tested eye. Patients may have abnormal aberrations that may create anomalous distributions for the probing light. If a narrow probing light beam enters the eye at locations with strong irregularity, it can cause problem in forming a compact probing light at the retina of the tested eye. In order to ensure to always obtain an acceptable wavefront measurement, a narrow probing beam can be moved within the pupil to a number of locations in the pupil. Only acceptable wavefront measurements are selected and averaged as the final wavefront measurement.

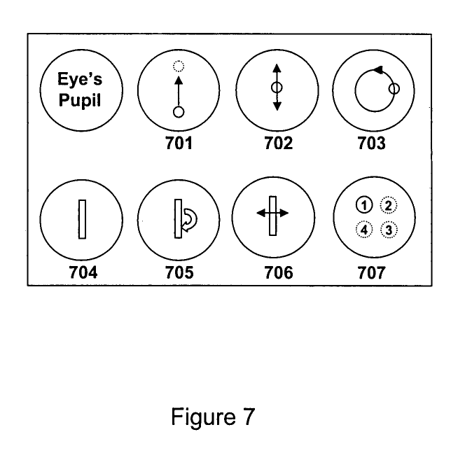

FIG. 7 shows the configurations of the movement of the probing light beam relative to the eye's pupil, including: a position-adjustable small beam 701, a translating and vertically scanning small beam across eye's pupil 702, a rotating small beam over the eye's cornea 703, a slit beam 704, rotating slit beam 705, a translating and laterally scanning slit beam 706, and an alternating small beams 707.

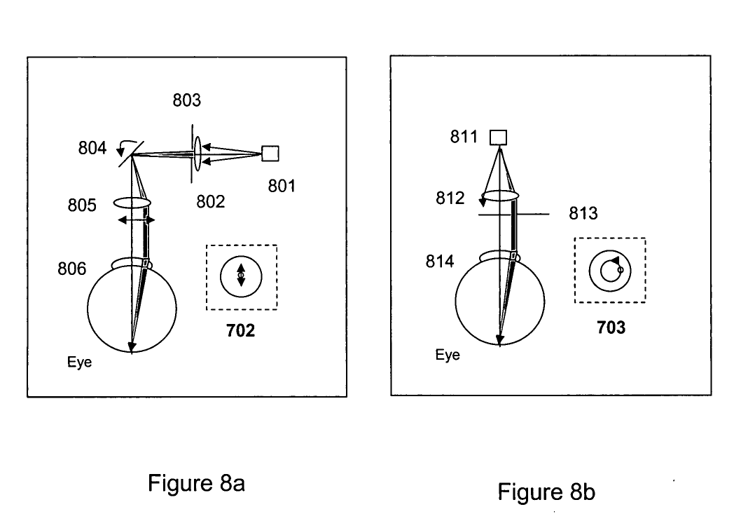

Many optical designs could be used to achieve these proposed configurations of probing beam. FIG. 8a shows the configuration 702 that scans a narrow probing light beam across the pupil in one direction. Light from a point light source 801 is imaged through a lens 802 and filtered by an aperture 803 to a cone-shaped narrow light beam. The narrow light beam is imaged on to a Galvo-scanner 804 that is at the focal plane of a positive lens 805. As the Galvo-scanner 804 rotates around its axis, it raster-scans the reflected narrow beam across the cornea of the patient's eye, as shown in the insertion 702.

FIG. 8b shows the arrangement for rotating a narrow probing light beam across the pupil. Light from a point light source 811 is expanded by the lens 812. A screen 813 with a small aperture is placed between the eye and the expanded beam. Rotating the screen 813 and the aperture creates a circularly moving light beam within the pupil of the eye, as shown in the insertion 703.

A number of embodiments have been described. Nevertheless, it will be understood that various modifications may be made without departing from the spirit and scope of the invention. For example, advantageous results still could be achieved if steps of the disclosed techniques were performed in a different order and/or if components in the disclosed systems were combined in a different manner and/or replaced or supplemented by other components. Accordingly, other embodiments are within the scope of the following claims.

What is claimed is:

1. A wavefront sensing system for determining the wave aberration of an eye, comprising:
a fixation target that is illuminated by a fixation light source at a location optically conjugate to the cornea of the eye, wherein the fixation target is partially visible by the eye without the need of a refractive correction and configured to keep the eye focus at its far accommodation point;
an illumination light source configured to produce a compact light source at the retina of the eye; and
a wavefront sensor configured to measure the outgoing wavefront originated from the compact light source at the retina of the eye to determine the wave aberration of the eye.

2. The wavefront sensing system of claim 1, wherein the fixation light source comprises a uniform beam and an aperture less than 2 mm in size positioned optically conjugated to the cornea of the eye.

3. The wavefront sensing system of claim 2, wherein the fixation light source comprises a light diffuser configured to receive a light illumination and to produce a uniform light illumination across the aperture.

4. The wavefront sensing system of claim 1, wherein the fixation light source comprises a light emitting diode.

5. The wavefront sensing system of claim 1, wherein the wavefront sensor is a Hartmann-Shack sensor.

6. The wavefront sensing system of claim 1, wherein the illumination light source is configured to produce a fixed divergent light beam across the pupil of the eye.

7. The wavefront sensing system of claim 1, further comprising a cylindrical lens configured to refract the outgoing wavefront originated from the retinal illumination and to transmit the refracted outgoing wavefront to the wavefront sensor.

8. The wavefront sensing system of claim 1, further comprising a non-contact sensor configured to automatically detect the left eye or the right eye in the wavefront measurement.

9. The wavefront sensing system of claim 1, further comprising a mechanism configured to sequentially move the illumination light source at multiple locations across the pupil of the eye.

10. A wavefront sensing system for determining the aberrations of an optical object having at least one optical surface, comprising:
an illumination light source configured to illuminate the optical object to produce a wavefront propagating from the object;
an optical system to relay the wavefront from the optical object to an plane;
a cylindrical lens configured to refract the wavefront at the plane; and
a Hartmann-Shack wavefront sensor having a lenslet array and a rectangular image sensor, configured to detect the refracted wavefront to determine the aberrations of the optical object.

11. A wavefront sensing system of claim 10, wherein the optical object is an human eye, and wherein the illumination light source is configured to produce a compact light source at the retina of the eye, and wherein the wavefront is the outgoing wavefront originated from the compact light source at the retina of the eye.

12. The wavefront sensing system of claim 11, wherein the cylindrical lens is positioned in front of the lenslet array of the Hartmann-Shack sensor to reduce the dimension of the wave sensing image alone one direction.

13. The wavefront sensing system of claim 11, wherein the cylindrical lens is positioned optically conjugate to the lenslet array of the Hartmann-Shack sensor.

14. A wavefront sensing system for determining the wave aberration of an eye, comprising:
an fixed divergent light beam through the pupil of the eye configured to produce a compact light source at the retina of the eye without an refractive correction for myopia, hyperopia or astigmatism of the eye; and
a wavefront sensor configured to detect the outgoing wavefront originated from the compact light source at the retina of the eye to determine the wave aberration of the eye.

15. The wavefront sensing system of claim 14, wherein the wavefront sensor is a Hartmann-Shack wavefront sensor.

16. The wavefront sensing system of claim 14, wherein the divergent light beam is produced by passing a collimated light beam through a negative spherical lens.

17. The wavefront sensing system of claim 14, wherein the divergent light beam is approximately −3 D at the corneal plane of the eye.

18. The wavefront sensing system of claim 14, wherein the wavefront error of the divergent light beam and the wavefront error of the eye in the illuminated pupil area is less than one half wavelength.

19. A wavefront sensing system for determining the wave aberration of an eye, comprising:
an illumination light source configured to produce a compact light source at the retina of the eye;
a wavefront sensor configured to detect the outgoing wavefront originated from the compact light source at the retina of the eye to determine the wave aberration of the eye; and
a non-contact sensor configured to automatically detect the left eye or the right eye during wavefront measurements.

20. The wavefront sensing system of claim 19, wherein the wavefront sensor is a Hartmann-Shack wavefront sensor.

21. The wavefront sensing system of claim 19, wherein the non-contact sensor includes a light source and a light detector.

22. A wavefront sensing system for determining the wave aberration of an eye, comprising:
an illumination light configured to illuminate a plurality of locations at the pupil of the eye to produce a compact light source at the retina of the eye sequentially; and
a Hartmann-Shack wavefront sensor configured to detect the outgoing wavefront originated from the compact light source at the retina of the eye to determine the wave aberration of the eye.

23. The wavefront sensing system of claim 22, wherein the illumination light is configured to move along a line, an arc, or a circle across the pupil of the eye.

24. The wavefront sensing system of claim 22, wherein the illumination light includes at least two light beams that can sequentially illuminate at two different locations at the pupil of the eye.

25. The wavefront sensing system of claim 22, wherein the Hartmann-Shack wavefront sensor is configured to detect a plurality of wavefront images from the outgoing wavefront originated from the compact light source at the retina of the eye.

26. The wavefront sensing system of claim 25, further comprising
a computer device configured to accept a wavefront image based on a predetermined image-quality criterion and to average a plurality of said accepted wavefront images to determine the wave aberration of the eye.
